(19) United States
(12) Patent Application Publication
(10) Pub. No.: US 2004/0092982 A1
(43) Pub. Date: May 13, 2004

(54) Medical instrument for use in cataract surgery and a method for use thereof

(76) Inventor: Yehiel Sheffer, Zichron Yaakov (IL)

Correspondence Address: LOWE HAUPTMAN GILMAN AND BERNER, LLP, 1700 DIAGONAL ROAD, SUITE 300 /310, ALEXANDRIA, VA, 22314, US

(21) Appl. No.: 10/297,322

(22) PCT Filed: Feb. 4, 2001
(86) PCT No.: PCT/IL01/00112

(30) Foreign Application Priority Data
Feb. 3, 2000 (IL) 134370

(51) Int. Cl. A61F 9/00

(52) U.S. Cl. 606/166

(57) ABSTRACT

The invention discloses a medical instrument, comprising: a handle, a blade portion having a sharpened edge for cutting a lens capsule of an eye at the periphery of a portion of said capsule to be removed during surgery, said blade portion being extendible and retractable from the handle tip portion thereby permitting insertion of said blade portion through a small incision in the eye when the blade is retracted and thereafter to correspond to a peripheral extent to said portion of the capsule to be removed during surgery, a mechanism for extending and retracting said blade portion, said mechanism located in said handle portion, and a foot pedal-controlled motor coupled to said handle for effecting cutting of capsule by sad blade portion. The invention additionally relates to a method for utilizing a medical instrument in performing cataract surgery.

### FIELD OF THE INVENTION

The present invention relates to a medical instrument for use in cataract surgery. More specifically, the present invention relates to a instrument having a retractable-extendible blade portion for use in an anterior capsulotomy during cataract surgery. The present invention also relates to a method for the use of said medical instrument in cataract surgery.

### BACKGROUND OF THE INVENTION

A cataract is a formation that occurs when the natural lens of the eye, responsible for focusing light and producing sharp images, becomes cloudy and hardens, resulting in a reduction of visual function. A cataract begins as a slight cloudiness in the lens that progressively grows more dense and opaque. As this occurs, the retina of the eye receives less and less light, and the light that does reach the retina is increasingly blurred and distorted. This causes gradual impairment of vision. If left untreated, blindness may result.

Modern advances in microsurgical techniques allow cataracts to be removed relatively safely and quickly, without the need for stitches. During cataract surgery, the cloudy lens of the eye is removed and, thereafter, an artificial lens is implanted in the eye for the restoring of vision.

To perform a cataract extraction, the surgeon first makes an incision in the cornea/sclera region of the eye. Carefully entering the eye through the incision, the surgeon then gently opens the front anterior portion of the capsule that encloses the lens (this step is referred to as a capsulotomy) and extracts the hard nucleus of the lens. The nucleus can either be extracted whole (a procedure commonly referred to as manual extracapsular cataract extraction), or it can be dissolved into tiny pieces through a technique called phacoemulsification (fragmentation by an ultrasonic oscillating probe), and thereafter, suctioned out of the eye.

The main advantage of the phacoemulsification technique over manual extraction of the nucleus is the small incision in the cornea/sclera needed to perform it. In manual cataract extraction, the surgical incision has to be about 7 millimeters wide, in order to allow for the nucleus to be removed whole. In phacoemulsification technique, because the nucleus is emulsified the surgical incision does not need to be as wide, and a 2-3 millimeter incision is sufficient.

A smaller incision is beneficial in regard to the rate of visual rehabilitation after operation as well as post-surgical astigmatism prevalence and severity.

In performing cataract extraction by phacoemulsification two mandatory demands have to be met. First, instruments used for intraocular manipulations during surgery should be small enough in order to account for their insertion through the small surgical wound (2-3 millimeters in diameter). Second, edges of the capsulotomy should be smooth and uniform. In contrast to whole nucleus extraction techniques, in which non-homogenous edges of the capsulotomy are acceptable, in phacoemulsification non-uniform edges of capsulotomy are unacceptable. The reason for this demand is that dug phacoemulsification process the presence of anterior capsular strands may result in capsular tear extending into the posterior capsule due to strands being captured and pulled by the suction unit A tear in the posterior capsule prevents the posterior lens implantation in most cases and might result in vitreous loss.

The most commonly used procedures for a capsulotomy include “can opener” capsulotomy and capsulorhexis. In a “can opener” capsulotomy, a specialized bent needle, known as an irrigated cytosome, is positioned on the lens capsule and used to perforate and tear a small triangular incision on the capsule. Thirty to forty of such incisions are made on the anterior capsule, such that the larger the number of incisions made, the more uniform and accurate the opening in the capsule will be. The capsule is caught by forceps and extracted upon completion of the capsulotomy.

While the “can opener” procedure is relatively easy to carry out and control, the resultant opening formed in the anterior capsule is comparatively ragged. As a result, there are often undesired radial openings produced at the tips of the triangular openings.

The capsulorhexis procedure was designed to ensure that a smooth edge is achieved at the anterior capsule. In a capsulorhexis, the surgeon opens a centrifugal linear opening in the capsule and then grips the anterior capsule edge with special forceps and creates a continuous circular tear of the anterior capsule.

While the circular capsulorhexis procedure has certain vantages, it is nonetheless a very delicate procedure that requires a high degree of expertise and surgical skill to perform. If the surgeon loses control on the tearing procedure, the tear might be directed posteriorly and cause opening in the posterior capsule with all its above mentioned complications. Furthermore, the circular capsulorhexis procedure is highly dependent on the convex morphology of the lens capsule, further detracting from the safety and efficacy of the procedure.

Accordingly, and in view of the ever-increasing incidence of cataracts, there has developed a need for a medical instrument and method for performing an anterior capsulotomy during cataract surgery which overcomes shortcomings and risks encountered using current techniques. There has also developed a need for an instrument for reliably and safely making an opening of the desired shape and diameter in the anterior capsule while not requiring such advanced precision and skill that is typically required for using instruments. DE4012882 relates to a surgical instrument for removing a cataract from the eye that has a circular blade which is rotated by a drive in the handle. However, the blade size is non-adjustable and therefore does not give the surgeon the ability to control the size of the cut in the anterior capsule.

### SUMMARY OF THE INVENTION

According to one aspect of the present invention there is provided a medical instrument for performing anterior capsulotomy procedures in cataract surgery comprising (a) a handle; (b) a blade portion having an adjustable diameter and having a sharpened edge for cutting a lens capsule at the periphery of the capsule to be removed during surgery, said blade portion being extendable and retractable from the handle portion so that the blade portion can be inserted, in the retracted form, through a small incision in the eye, and thereafter, extended to correspond to the size of the portion of the capsule to be removed during surgery, and subsequently retracted for allowing removal from the eye, wherein the blade portion can extend to a configuration having a 4-8 millimeter diameter range for cutting a lens capsule of an eye having a corresponding size; (c) a mechanism for extending and retracting said blade portion; (d) optionally, a foot pedal-controlled motor coupled to said handle for effecting cutting of capsule by said blade portion via slight clockwise and counterclockwise movements of the blade portion.

According to further features in the preferred embodiment of the invention described below, the handle has a proximal end and a distal end, said proximal end having all opening engaging the blade portion when said blade portion is extended.

According to further features in the preferred embodiment, the proximal end of the handle has a central rod protruding slidably therefrom having a probe with spikes for piercing the lens capsule, securing the instrument in place, and for catching the portion of the capsule membrane that is cut.

According to further features in the preferred embodiment the central rod has a blade attachment member attached thereto, said blade attachment member having a sharpened lower edge such that the portion of the capsule periphery at the proximal end of the handle not cut by the blade portion is cut by the blade attachment member.

According to further features in the preferred embodiment the blade portion comprises a flexible metal strip having a substantially flat sharpened lower edge.

According to further features in the preferred embodiment the blade portion comprises a flexible metal strip having a serrated lower edge such that cutting of capsule portion is achieved through applying gentle pressure to handle, thereby eliminating the need for the foot pedal-controlled motor.

According to further features in the preferred embodiment the metal strip is connected on both ends to a flexible belt, said metal strip and belt forming a continuous elongating loop through the handle and blade portion of the instrument.

According to further features in the preferred embodiment the flexible belt is made of nylon or any other suitable material.

According to further features in the preferred embodiment the loop is secured by a central chassis within the handle, and said loop is engaged, at one end, by a central gear within the handle.

According to further features in the preferred embodiment the central chassis is slidably disposed within the handle by two tracks extending longitudinally within the handle and by a retaining spring located at the distal end of the handle.

According to further features in the preferred embodiment the handle has graduations indicated on the exterior upper surface for extending the blade portion to the desired diameter.

According to further features in the preferred embodiment the tip of the handle can fit through an incision of approximately 3 millimeters in the cornea or sclera of the eye.

According to further features in the preferred embodiment the tip of the handle has a concave curvature corresponding to the shape of the incision in the cornea or sclera of the eye such that upon insertion of said handle tip through said incision, a temporary seal if formed around said incision preventing fluid loss from the eye.

According to further features in the preferred embodiment the mechanism for extending and retracting said blade portion includes a button translatably engaged to said handle such that translating said button to a specific extent causes said central chassis to slide accordingly, thereby regulating the degree to which blade portion is extended or retracted.

According to further features in the preferred embodiment the mechanism for extending and retracting said blade portion further includes a clutch drive, said clutch drive lowering and rotating when said button is pressed and moved, thereby causing rotating of central gear, and said rotating of central gear causing turning of said loop to expose the sharpened blade portion to the exterior in extent to the size of the periphery of the portion of the capsules to be removed during surgery.

According to further features in the preferred embodiment the handle is connected by a threaded coupling to a flexible mechanical cable, said cable fiber connecting said handle to a foot pedal-controlled motor.

According to further features in the preferred embodiment activating the foot pedal controlled-motor by pressing down briefly on the foot pedal causes the central gear to be engaged, thereby producing slight bidirectional movements in the loop to effect cutting of the lens capsule at the periphery of a portion of said capsule to be removed during surgery.

According to further features in the preferred embodiment the blade portion has a substantially circular or oval shape when extended.

In another aspect of the present invention, there is provided a method for utilizing a medical instrument in performing cataract surgery, comprising the steps of; (a) making an incision approximately 3 millimeters wide in the cornea of the eye; (b) inserting the handle tip through said incision; (c) extending the retracted blade portion to the required diameter, (d) positioning said extended blade portion in contact with the surface of an anterior lens capsule of said eye; (e) cutting said lens capsule at a peripheral extent of a portion of said lens capsule to be removed during said cataract surgery; (f) retracting said blade portion; (g) withdrawing said handle tip through said incision.

According to still further features in the preferred embodiment the step of cutting said lens capsule comprises gently applying pressure to the handle of the instrument, thereby causing cutting of said peripheral extent of the portion of said lens capsule by the blade portion.

According to still further features in the preferred embodiment the step of cutting said lens capsule comprises activating a foot pedal controlled motor by pressing down briefly on the foot pedal, thereby causing the central gear to be engaged and, subsequently, producing slight bidirectional movements in the loop causing cutting of the lens capsule at the periphery of a portion of said capsule by the blade portion.

According to still further features in the preferred embodiment the handle is approximately 17 centimeters in length and 1 centimeter in width at the distal end.

According to still further features in the preferred embodiment the opening at the proximal end of the handle is approximately 3 millimeters in width and 1.5-2.0 millimeters high.

According to still further features in the preferred embodiment the blade portion is 0.1-0.15 millimeters in thickness, 1.0-1.4 millimeters in width from the upper to lower edge, and 30-40 millimeters in length.

It is an object of the present invention to provide a medical instrument including an extendible-retractable blade portion which can be readily manipulated in size for minimizing the size of an incision required for its insertion in the eye, and which effects an uncomplicated and tearless cutting of the lens capsule to ensure reliable performance of a capsulotomy during cataract surgery.

The above and other objects, details, and advantages of the present invention will become apparent from the following detailed description, when read in conjunction with the accompanying drawings.

### BRIEF DESCRIPTION OF THE DRAWINGS

The invention is herein described, by way of example only, with reference to the accompanying drawings, wherein:

FIG. 1 is a partial cutaway perspective view of a instrument in accordance with the present invention with the blade portion in the extended form.

FIG. 2 is a sectioned side view of the handle of a medical instrument in accordance with the present invention.

FIG. 3 is a cross sectional view of the handle tip of a medical instrument in accordance with the present invention.

FIGS. 4a-c illustrate the blade portion of a medical instrument in accordance with the present invention.

FIG. 5 is a sectioned side view of the human eye during a surgical procedure employing the medical instrument in accordance with the present invention.

FIGS. 6 is a schematic drawing of a medical instrument coupled to a foot pedal-controlled motor according to the present invention.

### DETAILED DESCRIPTION OF THE DRAWINGS

It is to be understood that the medical instrument and methodology for using the same described herein and shown in the drawings represent a preferred embodiment of the present invention. Various modifications and additions may be made to the preferred embodiment without departing from the scope of the invention, as set out in the claims.

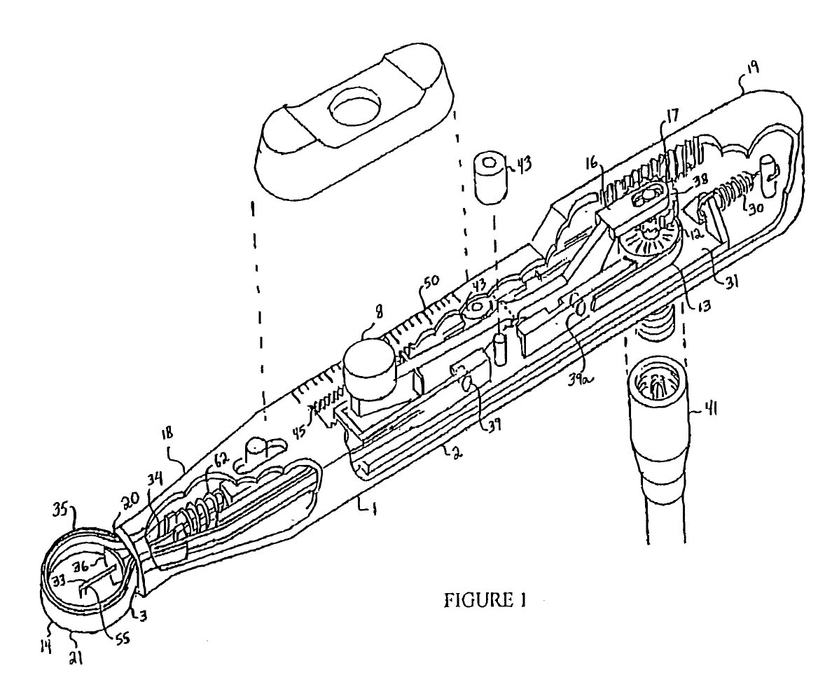

Referring to FIG. 1, medical instrument (1) of the present invention includes a handle (2) which is designed to be gripped by a surgeon, and a blade portion (3) (shown in FIG. 1 in the extended form) having a sharpened edge (21) for cutting a lens capsule of an eye at the periphery of a portion of said capsule to be removed during surgery. The handle (2) has a proximal end (18) and a distal end (19). Said handle (2) is approximately 17 centimeters in length and 10 millimeters in width at the distal end (19). The handle (2) has graduations (50) indicated on the exterior upper surface for facilitating extension of the blade portion (3) to the desired diameter. Said blade portion (3) is extendible and retractable from the opening (20) of the handle (2), so as to permit the insertion of the handle tip (when the blade portion (3) is retracted) through a small incision (approximately 3 millimeters wide) formed in the cornea or sclera of the eye. Thereafter, the blade portion (3) is extended to correspond precisely to the size of the portion of the capsule to be removed for surgery.

Medical instrument (1) further includes a mechanism for extending and retracting the blade portion (3). Said mechanism is located in the handle (2) and includes a button (8) translatably engaged by handle (2), such that translating said button (8) affects the degree to which the extendible-retractable blade portion is extended or retracted. The overall diameter of the blade portion (3), when extended, is dependent upon the diameter of the pupil of the particular eye upon which surgery is to be performed.

Medical instrument (1) further includes a threaded coupling (41) for connecting handle (2) with a foot pedal-controlled motor to effect cutting of a portion of a lens capsule by the blade portion (3) when said blade portion (3) is extended.

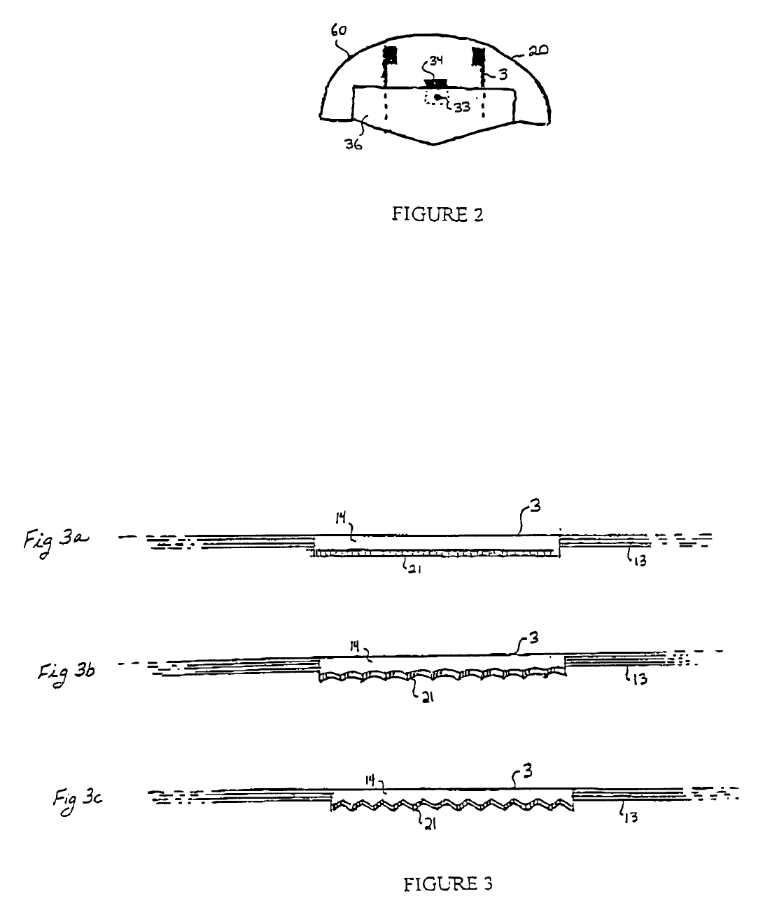

Referring now to FIG. 2, said handle has an opening (20) of about 3.5 millimeters in width and about 2.0 millimeters high at the proximal end of the handle from which the blade portion (3) extends. Said opening (20) has concave curvature (60) corresponding to the shape of the incision in the cornea or sclera of the eye such that upon insertion of said handle tip through said incision, a temporary seal if formed around said incision preventing fluid loss from the eye.

Referring now to FIG. 1 in combination with FIG. 2, extending from the opening (20) is a hollow central rod (34) having a probe (33) protruding slidably therefrom. Said probe (33) contains spikes (55) such that when blade portion (3) is extended, said spikes (55) function to secure the position on the lens capsule where the capsulotomy is to be performed and to catch the portion of the lens capsule that is cut in the capsulotomy. Said central rod (33) has a blade attachment member (36) attached thereto. Said blade attachment member (36) has a sharpened lower edge such that the portion of the capsule periphery not cut by the blade portion (3) is cut by the blade attachment member (36).

Referring now to FIGS. 3a-c, the blade portion (3) comprises a flexible metal strip (14) having a sharpened lower edge (21). Said sharpened lower edge (21) can be substantially flat, as shown in FIG. 3a, or serrated, as shown in FIGS. 3b and 3c. Blade portion (3) is 0.1-0.15 millimeters in thickness, 1.0-1.4 millimeters in width from the upper to lower edge, and 30-40 millimeters in length.

Referring now to FIG. 1 in combination with FIG. 3, the flexible metal strip (14) of the blade portion (3) is connected, by au adhesive or other suitable means, to a flexible belt (13). The flexible belt (13) can be made of nylon or any other suitable material and is approximately 300 millimeters in length. The flexible metal strip (14) and the flexible belt (13) together form a continuous elongating loop (35) through the instrument (1). Said loop (35) is secured by a central chassis (31) within the handle. Said loop (35) is engaged, at one end, by a central gear (12) at the distal end (19) of the handle (2). A pair of rollers (43) further secure the loop (35) in place. The central chassis (31) is slidably disposed within the handle (2) by two tracks (not shown) extending the lower length of the handle (2) and by a retaining spring (30) at the distal end (19) of the handle (2).

Referring again to FIG. 1, when the blade portion (3) is retracted (not shown), a small portion of said flexible belt (13) is exposed at the opening (20) of the handle (2), forming a small (approximately 2 millimeter diameter) circle, enabling the entry of the handle tip through the small incision of the cornea. To extend the blade portion (3), button (8) is translated proximally according to the graduations (50) on the handle (2). Said translation causes the central gear (12) to rotate (through a mechanism described below), thereby causing loop (35) to rotate, such that the exposed portion of said flexible belt (13) at the opening (20) of the handle (2) is replaced by the flexible metal strip (14) of the blade portion (3). Translation of button (8) also causes central chassis (31) to move forward thereby causing the diameter of said small circle to increase accordingly, and causing probe (33) to extend through blade attachment (36), as shown in FIG. 1. Probe spring (62) pulls the blade attachment member (36) towards the blade portion (3).

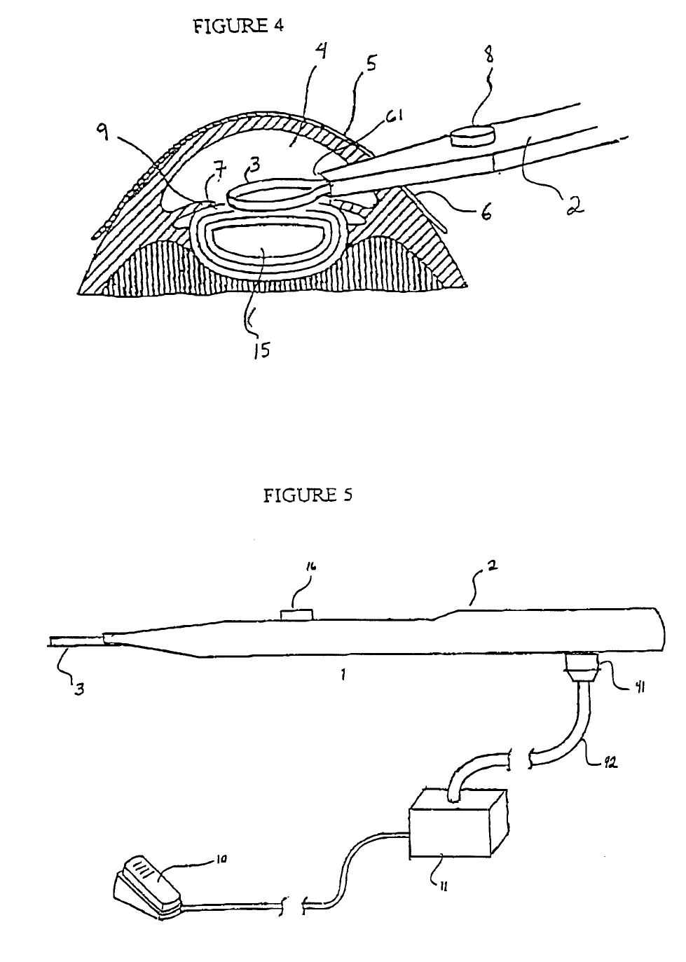

Referring now to FIG. 4 and to a method for use of a medical instrument in accordance with the present invention, a small incision (approximately 3 millimeters) is first made in the cornea (5) or sclera (6) of the eye and the handle tip (61) is inserted therethrough with the blade portion (3) fully retracted (not shown). Preferably, the incision in the cornea (5) or sclera (6) is made in a region where it will not effect the field of vision of the eye. Once inserted, the tip of the handle (61) forms a temporary seal around the incision, preventing the loss of fluid form the eye. The blade portion (3) is then extended by the surgeon to a diameter corresponding to the diameter of the portion of the anterior capsule (7) to be cut. Graduations on the upper surface of the handle permit the surgeon to set the blade diameter to the necessary extent. While the blade portion (3) can extend maximally to a configuration having a 4-8 millimeter diameter, it should be appreciated that it can be extended to any intermediate diameter as well. The handle tip (61) is aligned with the lens such that the handle tip (61) is approximately tangent to the outer surface of the lens (15). With the pupil dilated, the handle (2) is pushed down gently so as to secure the placement of the blade portion (3) over the anterior lens capsule (7). Pushing down gently on the handle (2) also causes the blade attachment member to cut the portion of the circular or oval region of the capsule that is not on the periphery of the blade portion (3) (i.e., the portion directly in front of the handle (2) opening). The anterior lens capsule (7) is then cut by the extended blade portion (3) forming a substantially perfect and uniform circular or oval cut in the anterior capsule (7). The surgeon can cut the capsule portion either by gently applying pressure to the handle (2) (in the case that said handle has a serrated blade), or by operating a foot pedal-controlled motor that is coupled to the handle (2) (in the case that said handle has a substantially flat blade), according to the arrangement illustrated in FIG. 5 and described below. After the cut has been made, the surgeon removes the cut portion of the anterior capsule (7) with the spikes. Cataract surgery is continued by phacoemulsification of the nucleus and aspiration of emulsified as well as soft cortical remnants, so as to leave only a clear posterior lens capsule within the eye to serve as a barrier between the anterior chamber and the vitreous cavity. In the case of extracapsular extraction, the capsulotomy is followed by nucleus removal and cortex aspiration by conventional techniques, however, the drawback of this procedure is the need for a larger incision.

Referring now to the arrangement in FIG. 5, the handle (2) has a connection to a threaded coupling (41), said threaded coupling (41) attached to a flexible mechanical cable (42) for coupling of the handle (2) to a foot pedal-controlled motor (11). In the embodiment in which the blade portion (3) has a substantially flat sharpened lower edge, (illustrated in FIG. 3a), the surgeon, to effect cutting of the lens capsule, presses down quickly and briefly on the foot pedal (10) pedal of the foot pedal-controlled motor (11). The activation of the motor (11) activates the central gear in the handle (2), thus producing slight bidirectional movements of 1-2 Hz frequency in the central gear and loop, and thereby causing cutting of the anterior lens capsule by the blade portion (3).

Alternatively, in the embodiment in which the blade portion has a serrated lower edge (as shown in FIGS. 3b, 3c), cutting of the anterior lens capsule is achieved through applying gentle pressure to handle (2), thereby eliminating the need for the foot pedal-controlled motor (11).

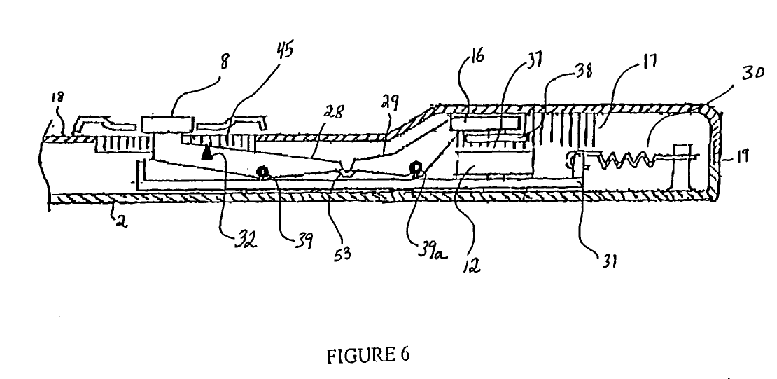

Referring now to FIG. 6, the mechanism for extending and retracting the blade portion of the medical instrument of the present invention includes a button (8) that is pressed inwardly and moved towards the proximal end (18) of the handle (2) to effect extension of the blade portion. A stopper (32) is attached to the side of a first lever (28) and connected to said button (8) such that pressing inwardly on the button (8) causes the stopper (32) to be released from an assembly of retaining notches (45) located along a region of the inner surface of the handle (2). Releasing of said stopper (32) permits the movement (either forward or backwards) of central chassis (31), the forward movement of central chassis caused by moving the button (8) in the proximal direction. Pressing inwardly on said button (8) furthermore causes upward shifting of a first lever (28) and a second lever (29) connected by internal hinge (53) to the first, said shifting occurring through two pivot points (39) (39a). Said upward shifting of first and second levers (28) (29) causes downward shifting of attachment member (16), thereby causing downward shifting of clutch drive (38) along central shaft (37) such that said clutch drive (38) becomes rotatably coupled to central gear (12). As central chassis (31) moves forward, clutch drive (38) is rotated along a gear-toothed surface (17) on the inner surface of the handle (2) thereby causing central gear (12) to rotate. As central gear (12) rotates, the loop (seen in FIG. 1) rotates to expose the blade portion of the loop to the exterior of the proximal end (18) of the handle (2) to replace the heretofore exposed flexible belt.

When the above described mechanism has been completely executed to achieve the desired diameter of extended blade portion (3), the button (8) is depressed at the forward position so as lock the central chassis (31) in place by causing the stopper (32) to be again fixed in the assembly of retaining notches (45) in the handle (2).

Preferably, the medical instrument of the present invention is formed so as to be disposable.

The medical instrument according to the present invention enjoys a number of advantages over the prior art. First, having an extendible-retractable blade portion the instrument according to the present invention requires a much smaller incision made in the cornea or sclera that commonly used instruments. Second, the diameter of the blade portion is size adjustable according to surgical needs and preferences. Finally, the extendible-retractable blade portion of the instrument according to the present invention reduces unwanted tearing of the lens capsule so that damage to the eye is less likely.

Although the present invention has been described in conjunction with a specific embodiment thereof, it is evident that many alternatives, modifications, and variations will be apparent to those skilled in the art. Accordingly, it is intended to embrace all such alternatives, modifications, and variations that fall within the spirit and scope of the claims.

1) A medical instrument, comprising;
a handle,
a blade potion having an adjustable diameter and having a sharpened edge for cutting a lens capsule of an eye at the periphery of a portion of said capsule to be removed during surgery, said blade portion being extendible and retractable from the handle tip portion thereby permitting insertion of said blade portion through a small incision in the eye when the blade is retracted and thereafter extended to correspond to a peripheral extent to said portion of the capsule to be removed during surgery, and subsequently retracted for allowing removal from the eye, wherein said blade portion can extend to a configuration having a 4-8 millimeter diameter range for cutting a lens capsule of an eye having a corresponding size;
a mechanism for extending and retracting said blade portion, said mechanism located in said handle portion, and
optionally, a foot pedal-controlled motor coupled to said handle for effecting cutting of the capsule by said blade portion via slight bidirectional movements of the blade portion.

2) A medical instrument according to claim 1 wherein the handle has a proximal end and a distal end, said proximal end having an opening engaging the blade portion when said blade portion is extended.

3) A medical instrument according to claim 2 wherein the proximal end of the handle has a central rod protruding slidably therefrom having a probe with spikes for piercing the lens capsule, securing the instrument in place, and for catching the portion of the capsule membrane that is cut.

4) A medical instrument according to claim 3 wherein the central rod has a blade attachment member attached thereto, said blade attachment member having a sharpened lower edge such that the portion of the capsule periphery at the proximal end of the handle not cut by the blade portion is cut by the blade attachment member.

5) A medical instrument according to claim 1 wherein the blade portion comprises a flexible metal strip having a substantially flat sharpened lower edge.

6) A medical instrument according to claim 1 wherein the blade portion comprises a flexible metal strip having a serrated lower edge such that cutting of capsule portion is achieved through applying gentle pressure to handle, thereby eliminating the need for the foot pedal-controlled motor.

7) A medical instrument according to claims 5 and 6 wherein the metal strip is connected to a flexible belt, said metal strip and belt forming a continuous elongating loop through the handle and blade portion of the instrument.

8) A medical instrument according to claim 7 wherein the flexible belt is made of nylon or any other suitable material.

9) A medical instrument according to claim 7 wherein the loop is secured by a central chassis within the handle, and said loop is engaged, at one end, by a central gear within the handle.

10) A medical instrument according to claim 9 wherein the central chassis is slidably disposed within the handle by two tracks extending longitudinally within the handle and by a retaining spring located at the distal end of the handle.

11) A medical instrument according to claim 1 wherein the handle has graduations indicated on the exterior upper surface for extending the blade portion to the desired diameter.

12) A medical instrument according to claim 1 wherein the tip of the handle can fit through an incision of approximately 3 millimeters in the cornea or sclera of the eye.

13) A medical instrument according to claim 1 wherein the tip of the handle has a concave curvature corresponding to the shape of the incision in the cornea or sclera of the eye such that upon insertion of said handle tip through said incision, a temporary seal if formed around said incision preventing fluid loss from the eye.

14) A medical instrument according to claim 10 wherein the mechanism for extending and retracting said blade portion includes a button translatably engaged to said handle such that translating said button to a specific extent causes said central chassis to slide accordingly, thereby regulating the degree to which blade portion is extended or retracted.

15) A medical instrument according to claims 7 and 14 wherein the mechanism for extending and retracting said blade portion further includes a clutch drive, said clutch drive rotating and lowering when said button is pressed, thereby causing rotating of central gear, and said rotating of central gear causing turning of said loop to expose the sharpened blade portion to the exterior in extent to the size of the periphery of the portion of the capsule to be removed during surgery.

16) A medical instrument according to claim 1 wherein the handle is connected by a threaded coupling to a flexible mechanical cable, said cable further connecting said handle to a foot pedal-controlled motor.

17) A medical instrument according to claim 16 wherein activating the foot pedal controlled-motor by pressing down briefly on the foot pedal causes the central gear to be engaged, thereby producing slight bidirectional movements in the loop to effect cutting of the lens capsule at the periphery of a portion of said capsule to be removed during surgery.

18) A medical instrument according to claim 1 wherein said blade portion has a substantially circular shape when extended.

19) A medical instrument according to claim 1 wherein said blade portion has a substantially oval shape when extended.

20) A method for utilizing a medical instrument in performing cataract surgery, comprising the steps of;
(a) making an incision approximately 3 millimeters wide in the cornea of the eye;
(b) inserting the handle tip through said incision;
(c) extending the retracted blade portion to the required diameter;
(d) positioning said extended blade portion in contact with the surface of an anterior lens capsule of said eye;
(e) cutting said lens capsule at a peripheral extent of a portion of said lens capsule to be removed during said cataract surgery;
(f) retracting said blade portion;
(g) withdrawing said handle tip through said incision.

21) A method according to claim 20 wherein said cutting comprises gently applying pressure to the handle of the instrument, thereby causing cutting of said peripheral extent of the portion of said lens capsule by the blade portion.

22) A method according to claim 21 20 wherein said cutting comprises activating a foot pedal controlled motor by pressing down briefly on the foot pedal, thereby causing the central gear to be engaged and, subsequently, producing slight bidirectional movements in the loop causing cutting of the lens capsule at the periphery of a portion of said capsule by the blade portion.

23) A medical instrument according to claim 2 wherein the handle is approximately 17 centimeters in length and 1 centimeter in width at the distal end.

24) A medical instrument according to claim 3 wherein the opening at the proximal end of the handle is approximately 3 millimeters in width and 1.5-2.0 millimeters high.

25) A medical instrument according to claim 1 wherein the blade portion is 0.1-0.15 millimeters in thickness, 1.0-1.4 millimeters in width from the upper to lower edge, and 30-40 millimeters in length.
